(19) United States
(12) Patent Application Publication
(10) Pub. No.: US 2014/0018586 A1
(43) Pub. Date: Jan. 16, 2014

(54) Method for Preparing Synthetic Natural Gas

(71) Applicants: Hyo Jun Lim, Guri-si (KR); Chang Dae Byun, Seoul (KR); Seok Jung Seo, Suwan-si (KR); Young Don Yoo, Seoul (KR); Do Yeon Lee, Seoul (KR); Mun Hyun Kim, Suwan-si (KR)

(72) Inventors: Hyo Jun Lim, Guri-si (KR); Chang Dae Byun, Seoul (KR); Seok Jung Seo, Suwan-si (KR); Young Don Yoo, Seoul (KR); Do Yeon Lee, Seoul (KR); Mun Hyun Kim, Suwan-si (KR)

(73) Assignee: POSCO, Pohang-si (KR)

(21) Appl. No.: 13/976,588

(22) PCT Filed: Dec. 27, 2011
(86) PCT No.: PCT/KR2011/010134

§ 371 Date: Jul. 30, 2013

(30) Foreign Application Priority Data
Dec. 28, 2010 (KR) 10-2010-0136837
Jan. 31, 2011 (KR) 10-2011-0009839

(51) Int. Cl. C07C 1/10

(52) U.S. Cl. CPC C07C 1/10
USPC 585/310; 422/162

(57) ABSTRACT

A method for preparing synthetic natural gas (SNG) is provided. More particularly, a method for preparing synthetic natural gas is provided, in which synthetic gas generated after fuel gasification and dust collection is subjected to a first methane synthesis reaction; only part of the gas is subjected to a water gas shift reaction and the remaining gas bypasses; the mixed gas prepared by mixing the gas passed through the water gas shift reaction and the gas bypassing the water gas shift reaction is subjected to a second methane synthesis reaction; and thereby heat of the methane synthesis reaction can be controlled and catalyst life can be lengthened.

### BACKGROUND

1. Field of the Invention

The present invention relates to a method for preparing synthetic natural gas and an apparatus for preparing synthetic natural gas.

2. Discussion of Related Art

Up until now, as a process for preparing synthetic natural gas (hereinafter referred to as “SNG”) that has reached a level of commercialization, a process for synthesizing sweet gas (that is, sweet gas that is purified to have less than 0.1 ppm of a concentration of a sulfuric compound, such as H₂S and COS in synthetic gas) using a Ni-based catalyst has been developed. Currently, since a commercialized desulfurization process is operated at room temperature or less, sweet gas that is emitted during a desulfurization process at room temperature or less is heated to at least 250° C. or more using a special heat exchanger in a process for synthesizing SNG and then supplied to a methanator (this is because a catalyst is activated at 250° C. or more, thereby processing methane synthesis).

A schematic diagram illustrating a conventional commercialized process for synthesizing SNG has been proposed as largely two steps as illustrated in FIGS. 1 and 2 attached below.

First, according to a process as illustrated in FIG. 1, synthetic gas obtained through coal gasification is passed through a heat recovery and dust collection process, and then adjusted to have a mole ratio of H₂/CO of about 3.0 through a water gas shift process. At this time, since a temperature of exhaust gas is 300° C. or more, the temperature of synthetic gas should be decreased to room temperature in view of properties of a desulfurization process that is operated at room temperature or less. Sulfuric compounds (H₂S and COS) and CO₂ included in synthetic gas are removed at room temperature or less, and then the resulting synthetic gas is supplied to a process for synthesizing SNG. At this time, the synthetic gas supplied to the process for synthesizing SNG is pre-heated from room temperature to 250° C. or more through a proper heat exchanging method, and then supplied to a methanator in the process for synthesizing SNG.

In addition, according to a process of performing methane synthesis and a water gas shift reaction at the same time as illustrated in FIG. 2, synthetic gas obtained through coal gasification is cooled and dust-collected through a heat exchanger, cooled to room temperature or less, and then supplied to a desulfurization process. Sweet gas emitted from the desulfurization process is pre-heated to 250° C. or more through a proper heat exchange, supplied to a reactor filled with a catalyst capable of performing a methane synthesis reaction and a water gas shift reaction at the same time, and thereby SNG including CH₄ and CO₂ as main components can be obtained. In such a case, since CO₂ is generated by the water gas shift reaction, a process for removing CO₂ is needed at the rear end. At this time, a catalyst capable of performing the methane synthesis reaction and water gas shift reaction at the same time is used. In other words, the water gas shift process and the methane synthesis process need to be separately constituted or performed at the same time by developing a catalyst.

In addition, although it is not yet commercialized, Korean Patent Application No. 2010-116540 suggests that since it is known that a Ce—Mo-based catalyst is suitable for a methane-synthesizing catalyst on synthetic gas including a sulfuric compound, a process for preparing SNG without a cooling and heating process can be performed using such a catalyst. In other words, as illustrated in FIG. 3, a method including precipitating high-temperature synthetic gas supplied from a gasifier and passing the gas through a dust collection and water gas shift process, performing a water gas shift process at a temperature of 300° C. or more, supplying the gas to a methane synthesis process to minimize an unnecessary heat exchanging process, and then performing a desulfurization and CO₂-removing process at the rear end of the methane synthesis process is suggested. Even in this case, there is a problem in that different catalysts suitable for each sulfur-containing synthetic gas should be used.

### SUMMARY OF THE INVENTION

Accordingly, the present inventors focused their efforts to solve the problems described above, and as a result, developed a method and an apparatus for preparing synthetic natural gas, in which the method is capable of controlling heat of a reaction for methane synthesis and lengthening catalyst life. Consequently, the present invention was completed.

Accordingly, an object of the present invention is to provide a method for preparing synthetic natural gas, in which synthetic gas generated after fuel gasification and dust collection is subjected to a first methane synthesis reaction; then only part of the gas is subjected to a water gas shift reaction, the remaining gas bypasses, and then all the gas is mixed; and then the mixed gas is again subjected to a second methane synthesis reaction; and thereby the method is capable of controlling heat of a reaction for methane synthesis and lengthening catalyst life.

Another object of the present invention is to provide an apparatus for preparing the synthetic natural gas.

As a method for solving the above objects, an aspect of the present invention provides a method for preparing synthetic natural gas (SNG), the method including:

subjecting synthetic gas generated after fuel gasification and dust collection in a saturated state mixed with steam to a first methane synthesis reaction (Step 1); and

subjecting part of synthetic gas emitted from the first methane synthesis reaction to a water gas shift reaction and bypassing the remaining gas without subjecting it to the water gas shift reaction, and then subjecting the mixed gas mixed of the bypassed synthetic gas and the synthetic gas emitted from the water gas shift reaction to a second methane synthesis reaction (Step 2).

As a method for solving the above object, another aspect of the present invention provides an apparatus for preparing synthetic natural gas (SNG), the apparatus including:

a saturating unit for mixing steam and synthetic gas generated after fuel gasification and dust collection;

a first methane synthesis reaction unit for performing a methanation reaction of saturated gas generated from the saturating unit;

a water gas shift reaction unit for performing a water gas shift reaction of part of the synthetic gas emitted from the first methane synthesis reaction unit;

a bypassing unit for bypassing the remaining synthetic gas emitted from the first methane synthetic reaction unit without passing it through the water gas shift reaction unit; and

a second methane synthesis reaction unit for performing a second methanization reaction of the mixed gas, which is prepared by mixing the synthetic gas emitted from the water gas shift reaction unit and the synthetic gas of the bypassing unit.

### BRIEF DESCRIPTION OF THE DRAWINGS

The above and other objects, features and advantages of the present invention will become more apparent to those of ordinary skill in the art by describing in detail exemplary embodiments thereof with reference to the attached drawings, in which:

FIGS. 1 and 2 illustrate a process for preparing SNG using sweet gas after desulfurization;

FIG. 3 illustrates a process for preparing SNG from coal using sour gas before desulfurization;

FIG. 4 illustrates a process for preparing SNG according to an embodiment of the present invention; and

FIG. 5 illustrates a process for preparing SNG according to an embodiment of the present invention.

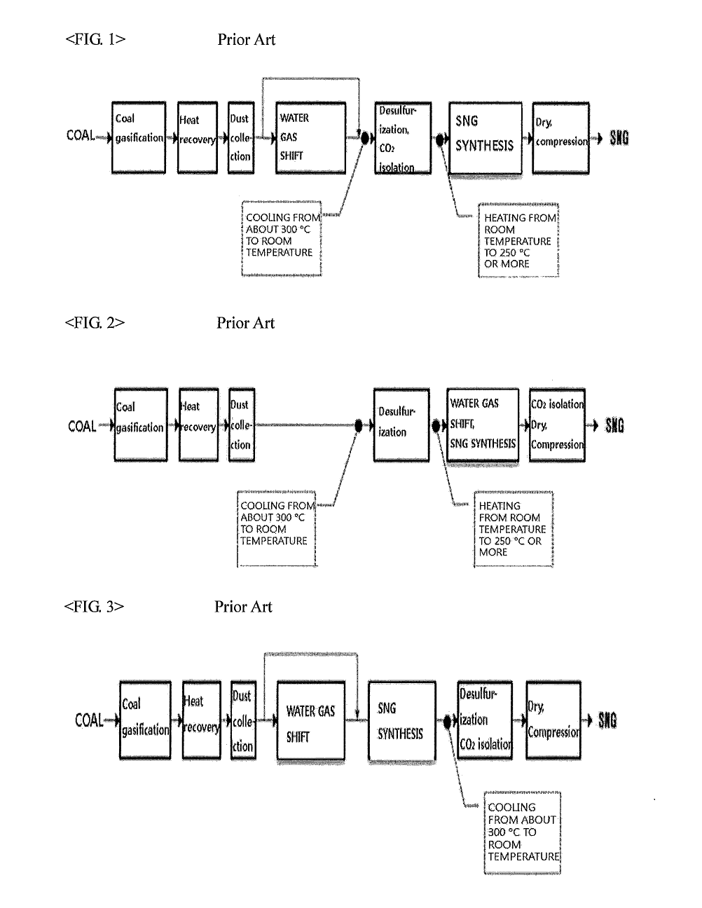

### DETAILED DESCRIPTION OF EXEMPLARY EMBODIMENTS

Hereinafter, exemplary embodiments of the present invention will be described in detail. However, the present invention is not limited to the embodiments disclosed below, but can be implemented in various forms. The following embodiments are described in order to enable those of ordinary skill in the art to embody and practice the present invention.

The present invention relates to a method for preparing synthetic natural gas (SNG), the method including subjecting synthetic gas generated after fuel gasification and dust collection in a saturated state mixed with steam to a first methane synthesis reaction (Step 1); and subjecting part of synthetic gas emitted from the first methane synthesis reaction to a water gas shift reaction and by-passing the remaining gas without subjecting it to the water gas shift reaction, and then subjecting the mixed gas mixed of the bypassed synthetic gas and the synthetic gas emitted from the water gas shift reaction to a second methane synthesis reaction (Step 2).

The fuel is a hydrocarbon-based raw material, for example, coal, biomass, waste matter, vacuum residue, and the like, but the present invention is not limited thereto.

In general, the synthetic gas in Step 1 is a synthetic gas used for a method for preparing natural gas, and may be sweet gas that is subjected to fuel gasification, a dust collection process, and additionally a desulfurization process and may also be sulfur-containing sour gas that is subjected to the fuel gasification and dust collection process, but not subjected to the desulfurization process. In addition, it is preferable that the synthetic gas satisfy a condition of a mole rate of H₂/CO of 0.5 to 1.0.

In addition, a method for preparing SNG according to the present invention may be performed in a single reactor, and one process for preparing SNG may be performed in multiple reactors.

In addition, a method for preparing SNG according to the present invention may further include a methane synthesis reaction after a second methane synthesis reaction.

Hereinafter, the present invention will be described in more detail.

Main reaction formulas of a water gas shift reaction and a methane synthesis reaction for preparing SNG are as follows:

Methane synthesis reaction formula: CO+3H₂→CH₄+H₂O (Heat of reaction: 206 kJ/mol)

Water gas shift reaction formula: CO+H₂O→H₂+CO₂ (Heat of reaction: 41 kJ/mol)

Since synthetic gas supplied in a first methane synthesis reaction has a mole ratio of H₂/CO of 1.0 or less (preferably, 0.5 to 1.0), as compared with the case of using synthetic gas having a mole ratio of H₂/CO of 3.0 as usual, heat of the reaction is low and water is present in the synthetic gas in a saturated state, so that temperature rise in a catalyst layer is low and thus a catalyst sintering problem that becomes a problem at 700° C. or more can be solved. In this way, since the synthetic gas obtained after performing the first methane synthesis reaction still has a low concentration of CH₄ and non-shifted CO and H₂ are present, only some of the gas emitted from the first methane synthesis reaction is subjected to the water gas shift reaction, the remaining gas bypasses the water gas shift reaction, the gas that is subjected to the water gas shift reaction and the gas that bypasses the water gas shift reaction are mixed, and then the mole ratio of H₂/CO in the mixed gas is adjusted to 2.7 to 3.3 (preferably 2.9 to 3.1). At this time, cooling the synthetic gas emitted from the first methane synthesis reaction to 300 to 350° C. may be further included. In this way, the mixed gas (the gas being supplied in a second methane synthesis reaction) prepared by mixing the gas passed through the water gas shift process and the gas that bypasses the water gas shift process is composed of a CH₄ component, a CO component, a H₂ component (maintaining a mole ratio of H₂/CO of 2.7 to 3.3), CO₂ and H₂O produced from the water gas shift reaction. The gas having such a composition is subjected to the second methane synthesis reaction again. The second methane synthesis reaction may be performed by repeating the methane synthesis reaction more than once (when using a single reactor, the single reactor includes a second methane synthesis reaction catalyst layer, a third methane synthesis reaction catalyst layer, a fourth methane synthesis reaction catalyst layer, and the like, and when using multiple reactors, the multiple reactors includes a second methanator, a third methanator, a fourth methanator, and the like).

In addition, the synthetic gas emitted from the first methane synthesis reaction should be able to be further cooled to 280 to 320° C. so as to prevent excessive temperature rise in a second methanator that is connected at the rear end before the water gas shift reaction. In addition, an amount of the gas supplied in the water gas shift reaction may be controlled through a bypass valve, thereby adjusting a mole ratio of H₂/CO in the gas supplied in the second methane synthesis reaction to be 2.7 to 3.3.

In addition, the present invention relates to an apparatus for preparing synthetic natural gas (SNG), in which the apparatus includes:

a saturating unit for mixing steam and synthetic gas generated after fuel gasification and dust collection;

a first methane synthesis reaction unit for performing a methanation reaction of saturated gas generated from the saturating unit;

a water gas shift reaction unit for performing a water gas shift reaction of part of the synthetic gas emitted from the first methane synthesis reaction unit;

a bypassing unit for bypassing the remaining synthetic gas emitted from the first methane synthetic reaction unit without subjecting it to the water gas shift reaction unit; and

a second methane synthesis reaction unit for performing a second methanation reaction of the mixed gas, which is prepared by mixing the synthetic gas emitted from the water gas shift reaction unit and the synthetic gas of the bypassing unit.

The methane synthesis unit and water gas shift reaction unit may be included in a single reactor, or each of the units may be included in a single reactor, thereby performing one process in multiple reactors.

The methane synthesis reaction units may further be included continuously at the rear end of the second methane synthesis reaction unit.

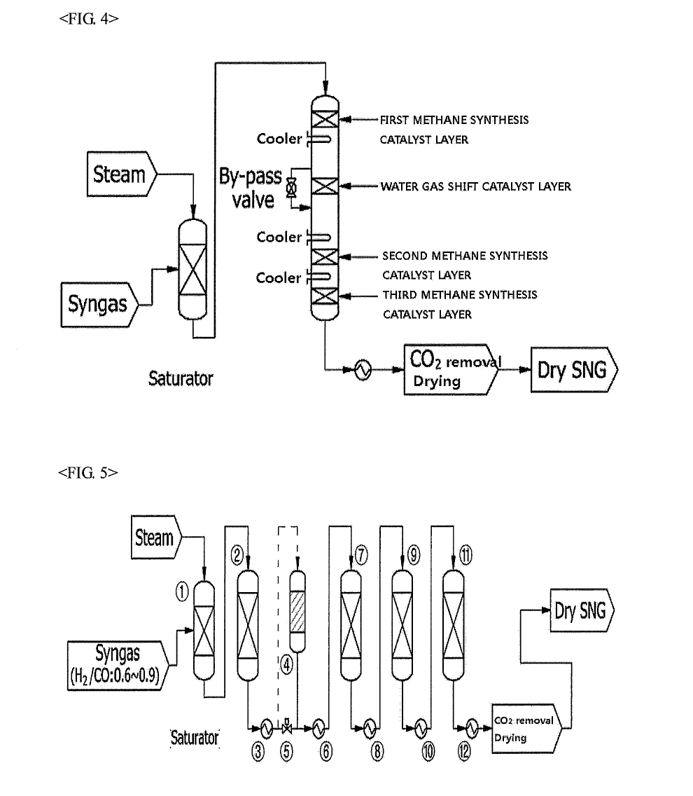

A schematization of an example of the case of using a single reactor as described above is illustrated in FIG. 4.

FIG. 4 illustrates a process for preparing SNG, in which one reactor includes a methane synthesis reaction catalyst layer, a water gas shift reaction catalyst layer, and a plurality of methane synthesis reaction catalyst layers. In other words, the synthetic gas emitted from a gasifier is cooled to a proper temperature through heat recovery, dust-collected, desulfurized (a mole ratio of H₂/CO is still 0.5 to 1.0), and then supplied to a saturator mixing steam and synthetic gas. Water in the synthetic gas is saturated in the saturator, pre-heated to 250° C. or more, and then supplied to the methane synthesis reaction catalyst layer. In such a case, the temperature of synthetic gas emitted from the methane synthesis reaction catalyst layer is not more than 700° C. This is because the water is saturated and a mole ratio of H₂/CO is 0.5 to 1.0, and thereby heat of methane synthesis reaction is not high. For the gas emitted from the first methane synthesis reaction catalyst layer, after being cooled to between 300 and 350° C., part of the gas is supplied to the water gas shift reaction catalyst layer for the water gas shift reaction and the remaining gas is not subjected to the water gas shift reaction catalyst layer through a bypass valve. The water gas shift reaction catalyst layer includes the bypass valve, so that by controlling a bypass flow rate, the gas passed through the water gas shift catalyst layer and the gas that bypasses through the bypass valve are mixed at a mole ratio of H₂/CO of 2.7 to 3.3 (preferably, 2.9 to 3.1). In other words, the mole ratio of H₂/CO in the mixed gas prepared by mixing the gas passed through the water gas shift reaction catalyst layer and the gas supplied through the bypass valve is adjusted to be 2.7 to 3.3 (preferably, 2.9 to 3.1). When the mole ratio of H₂/CO in the synthetic gas emitted from the first methane synthesis reaction catalyst layer is not within such a range, the whole quantity of the gas emitted from the first methane synthesis reaction catalyst layer bypasses, so that the gas is not supplied to the water gas shift process. Since the water gas shift reaction is an exothermic reaction, the discharge gas is again cooled to 280 to 320° C. by installing a cooler, and then again supplied to a second or third methane synthesis reaction catalyst layer to have a required CH₄ concentration (specifically, 96 to 99%).

It is preferable that the methane synthesis be performed under a Ni-based catalyst when methane synthesis is performed using sweet gas without sulfur, and that the methane synthesis be performed under a Ce—Mo-based catalyst when sour gas before desulfurization is used. However, the present invention is not limited thereto.

In addition, the water gas shift reaction is preferably performed under a Co—Mo-based catalyst, but the present invention is not limited thereto.

In addition, a schematization of an example of the case of performing in multiple reactors is illustrated in FIG. 5. A concept of the catalyst layer illustrated in FIG. 4 is constituted of each independent reactor to cool easily. The synthetic gas passed through a saturator 1 is passed through a first methanator 2, and then cooled to 300 to 350° C. Then, part of the gas is passed through a water gas shift reactor 4, while the remaining gas bypasses, and then mixed with the gas passed through the water gas shift reaction at the rear end of the water gas shift reactor. When the gas is mixed, a mole ratio of H₂/CO in the gas should be 2.7 to 3.3 (preferably, 2.9 to 3.1). When a mole ratio of H₂/CO in the synthetic gas emitted from the first methanator is not within such a range, the total amount of the gas emitted from the first methanator bypasses, and thus the gas is not supplied to the water gas shift process. The gas emitted from the water gas shift reactor 4 should be passed through the second and third methanators to form SNG having a required CH₄ concentration. In other words, the CH₄ concentration in SNG is possibly adjusted by controlling the number of the methanators.

Such a process may be applied to sweet gas passed through a dust collection and desulfurization process, but also applied without change to sour gas only passed through a dust collection process. Instead a catalyst capable of being applied to the synthetic gas including a sulfur component should be used.

Hereinafter, the present invention will be described in more detail with reference to Examples. However, the following Examples are only for illustrating the present invention, and the content of the present invention is not intended to be limited to the following Examples.

### Example 1

### Preparation of SNG Using Multiple Reactors (FIG. 5)

The following Table 1 shows a gas composition at each site of the multiple reactors (FIG. 5).

First, as a flow rate of synthetic gas, 1000 Nm³/h (a supply ratio of H₂/CO is 0.93) was supplied at 41 atmospheric pressure and a supply flow rate of steam was 512 Nm³/h. There is especially an advantage in that CO₂ in the synthetic gas supplied from a gasifier may not be separately removed in advance and supplied to a first methanator. The CH₄ concentration at the rear end of the first methanator was about 15.7% and the reaction temperature was 698° C. Only part (12%) of the gas generated at an exit of the first methanator was supplied to the water gas shift reactor (at this time, the reactor was cooled to about 320° C.), the remaining gas (88%) was bypassed, then mixed with the gas emitted from the water gas shift reactor, and then supplied to a second methanator. At this time, it could be confirmed from the composition of an inlet of the second methanator that a mole ratio of H₂/CO was 3.0. This mixed gas was supplied to the second methanator and then the second methane synthesis reaction was performed. It could be confirmed that the CH₄ concentration at the exit of the second methanator was about 28.2% and the reaction temperature was 533° C. When the CH₄ concentration at an exit of a third methanator 3 was 34.4% and 99% of CO₂ was removed, the finally obtained concentration of SNG was about 97%.

TABLE 1

| | Supplies | | Rear end of | Inlet of | Exit of | Exit of | |
|---|---|---|---|---|---|---|---|
| | synthetic | | first | second | second | third | Dry |
| Items | gas | Steam | methanator | methanator | methanator | methanator | SNG |
| Temperature | 280 | 254 | 698.2 | 297.6 | 532.6 | 351.9 | 25 |
| (° C.) | | | | | | | |
| Pressure (bar) | 40 | 43 | 40 | — | 40 | 40 | 40 |
| Mole Flow | 44.6 | 22.8 | 48.208 | 48.208 | 44.184 | 42.888 | 15.989 |
| (kmol/hr) | | | | | | | |
| Volume Flow | 1,000 | 512 | 1,080 | 1,080 | 990 | 961 | 358 |
| (Nm³/h) | | | | | | | |

Mole %(dry)

| | | | | | | | |
|---|---|---|---|---|---|---|---|
| CO | 37.5 | — | 10.9 | 9.7 | 1.7 | 0.0 | 0.1 |
| H₂ | 35.0 | — | 28.2 | 29.0 | 11.3 | 1.8 | 0.2 |
| CO₂ | 22.2 | — | 44.8 | 45.4 | 58.3 | 63.3 | 1.4 |
| CH₄ | 4.9 | — | 15.7 | 15.5 | 28.2 | 34.4 | 96.9 |
| H₂O | — | 100.0 | — | — | — | — | — |
| N₂ | 0.3 | — | 0.3 | 0.3 | 0.3 | 0.4 | 1.4 |
| H₂/CO ratio | 0.9 | — | 2.6 | 3.0 | — | — | — |

According to a method for preparing SNG of the present invention, synthetic gas (having a mole ratio of H₂/CO of 0.5 to 1.0) emitted from a gasifier is first subjected to a methane synthesis reaction without being subjected to a water gas shift reaction to reduce calories generated from the methane synthesis reaction and thereby suppress a sharp increase in temperature of a catalyst layer, so that catalyst life can be lengthened.

In addition, in the present invention, both sweet gas that is subjected to dust collection and desulfurization processes and sour gas that is subjected to a dust collection process but not subjected to a desulfurization process can be used.

In addition, according to the present invention, a synthetic gas recirculation system for controlling temperature is unnecessary, so that the operation is easy and construction expenses can significantly be reduced.

While the invention has been shown and described with reference to certain exemplary embodiments thereof, it will be understood by those skilled in the art that various changes in form and details may be made therein without departing from the spirit and scope of the invention as defined by the appended claims.

### BRIEF DESCRIPTION OF PARTS IN THE DRAWINGS

1. Saturator
2. Methanator 1
3. Cooler 1
4. Water gas shift reactor
5. Bypass valve
6. Cooler 2
7. Methanator 2
8. Cooler 3
9. Methanator 3
10. Cooler 4
11. Methanator 4
12. Cooler 5

What is claimed is:

1. A method for preparing synthetic natural gas (SNG), the method comprising:
subjecting synthetic gas generated after fuel gasification and dust collection in a saturated state mixed with steam to a first methane synthesis reaction (Step 1); and
subjecting part of synthetic gas emitted from the first methane synthesis reaction to a water gas shift reaction and bypassing the remaining gas without subjecting it to the water gas shift reaction, and then subjecting the mixed gas mixed of the bypassed synthetic gas and the synthetic gas emitted from the water gas shift reaction to a second methane synthesis reaction (Step 2).

2. The method of claim 1, wherein the fuel is a hydrocarbon-based raw material.

3. The method of claim 1, wherein a mole ratio of H₂/CO in the synthetic gas of Step 1 is 0.5 to 1.0.

4. The method of claim 1, further comprising performing a methane active reaction after the second methane synthesis reaction.

5. The method of claim 1, further comprising a desulfurization process after the dust collection.

6. The method of claim 1, further comprising cooling the synthetic gas emitted from the first methane synthesis reaction to 300 to 350° C.

7. The method of claim 1, further comprising cooling the synthetic gas emitted from the water gas shift reaction to 280 to 320° C.

8. The method of claim 1, wherein, in the synthetic gas at the time of performing the second methane synthesis reaction, a mole ratio of H₂/CO is 2.7 to 3.3.

9. The method of claim 1, which is performed in one reactor or in one process.

10. An apparatus for preparing synthetic natural gas (SNG), the apparatus comprising:
a saturating unit for mixing steam and synthetic gas generated after fuel gasification and dust collection;
a first methane synthesis reaction unit for performing a methanation reaction of saturated gas generated from the saturating unit;
a water gas shift reaction unit for performing a water gas shift reaction of part of the synthetic gas emitted from the first methane synthesis reaction unit;
a bypassing unit for bypassing the remaining synthetic gas emitted from the first methane synthetic reaction unit without passing it through the water gas shift reaction unit; and
a second methane synthesis reaction unit for performing a second methanization reaction of the mixed gas, which is prepared by mixing the synthetic gas emitted from the water gas shift reaction unit and the synthetic gas of the bypassing unit.

11. The apparatus of claim 10, further comprising a cooler at the rear end of the methane synthesis reaction unit.

12. The apparatus of claim 10, further comprising a cooler at the rear end of the water gas shift reaction unit.

13. The apparatus of claim 10, wherein the synthetic gas supplied to the saturating unit is a synthetic gas passed through a desulfurization process after dust-collecting.
